(19) United States
(12) Patent Application Publication
(10) Pub. No.: US 2026/0232991 A1
(43) Pub. Date: Aug. 13, 2026

(54) METHOD FOR DETERMINING A FLUID VOLUME FLOW THROUGH AN IMPLANTED VASCULAR SUPPORT SYSTEM AND VASCULAR SUPPORT SYSTEM

(71) Applicant: KARDION GMBH, Stuttgart (DE)

(72) Inventors: Hardy Baumbach, Stuttgart (DE); Julian Kassel, Böblingen (DE); Inga Schellenberg, Stuttgart (DE); Martina Budde, Karlsruhe (DE); Thomas Alexander Schlebusch, Renningen (DE)

(21) Appl. No.: 19/383,423

(22) Filed: Nov. 7, 2025

Related U.S. Application Data
(63) Continuation of application No. 15/734,004, filed on Jun. 6, 2019, now Pat. No. 12,491,357.

(30) Foreign Application Priority Data
Jun. 6, 2018 (DE) 102018208870.5

(51) Int. Cl. A61M 60/523; A61M 60/178; A61M 60/216; A61M 60/515; A61M 60/546; A61M 60/857

(52) U.S. Cl. CPC A61M 60/523; A61M 60/178; A61M 60/216; A61M 60/515; A61M 60/546; A61M 60/857; A61M 2205/3368; A61M 2205/3653

(57) ABSTRACT

The invention relates to a method for determining a fluid volume flow (1) through an implanted vascular support system (2), comprising the following steps: a) determining a fluid temperature parameter in the region of a cannula (4) of the support system (2), b) operating a heating element (5) which can bring about a change in a fluid temperature in the cannula (4), c) determining the fluid volume flow (1) using at least the fluid temperature parameter or the change thereof and at least one heating element operating parameter or the change thereof. The invention also relates to a vascular support system.

The invention relates to a method for determining a fluid volume flow through an implanted vascular support system, a processing unit, and an implantable vascular support system. The invention is in particular used in (fully) implanted left-heart support systems (LVAD).

Implanted left-heart support systems (LVAD) mainly exist in two design variants. (Percutaneous) minimally invasive left-heart support systems constitute a first common design variant. Apical left-heart support systems invasively implanted under the chest opening constitute the second common design variant. In the first variant mentioned, blood is conveyed directly from the left ventricle into the aorta since the (percutaneous) minimally invasive left-heart support system is positioned centrally in the aortic valve. In the second variant mentioned, the blood is apically conveyed from the left ventricle via a bypass tube into the aorta.

The task of a cardiac support system is to convey blood. In this case, the so-called heart-time volume (HTV, usually indicated in liters per minute) is highly clinically relevant. In other words, the heart-time volume in this case relates to the total volume flow of blood (from a ventricle), in particular from the left ventricle, to the aorta. Correspondingly clear is the attempt to collect this parameter as a measured value during operation of a cardiac support system.

Depending on the level of support, which describes the proportion of volume flow conveyed by a conveying means, such as a pump of the support system, to the total volume flow of blood from the ventricle to the aorta, a certain volume flow reaches the aorta via the physiological path through the aortic valve. The heart-time volume or the total volume flow (Q_{HTV}) from the ventricle to the aorta is therefore usually the sum of the pump volume flow (Qₚ) and the aortic valve volume flow (Qₐ). This can be expressed with the following relationship:

$${Q}_{HTV} = {Q}_{p} + {Q}_{a}$$

An established method for the determination of the heart-time volume (Q_{HTV}) in the clinical setting is the use of dilution methods, which, however, all rely on a catheter inserted transcutaneously and therefore can only provide heart-time volume measurement data during cardiac surgery. Since the detection of the heart-time volume (Q_{HTV}) through an LVAD is difficult to implement, Qₚ can be detected by suitable components of the LVAD. For high levels of support (i.e., Qₚ/Q_{HTV}), Qₐ approaches zero so that Qₚ can approximately be used as the heart-time volume (Q_{HTV}).

An established method for measuring the pump volume flow (Qₚ) is the correlation of the operating parameters of the support system, predominantly the electrical power consumption, possibly supplemented by further physiological parameters, such as blood pressure. Since these methods are based on statistical assumptions and the underlying pump characteristic map of the LVAD used, the correlated Qₚ are error-prone. In order to increase the measurement quality of the parameter Qₚ, the inclusion of a flow sensor is therefore desirable.

The object of the invention is to specify an improved method for determining a fluid volume flow in the region of an implanted vascular support system and to create an improved implantable vascular support system.

In particular, it is an object of the invention to specify a method for determining a fluid volume flow in the region of an implanted vascular support system and to create an implantable vascular support system by means of which a fluid volume flow in a blood flow region can be determined in a human or animal body, in which the vascular support system is implanted or arranged.

According to claim 1, a method for determining a fluid volume flow through an implanted vascular support system is proposed here, comprising the following steps:
a) determining a fluid temperature parameter in the region of a cannula of the support system,
b) operating a heating element which can bring about a change in a fluid temperature in the cannula,
c) determining the fluid volume flow using at least the fluid temperature parameter or the change thereof and at least one heating element operating parameter or the change thereof.

The vascular support system is preferably a cardiac support system, particularly preferably a ventricular support system. The method preferably serves to determine a fluid volume flow through a blood vessel or through a cross-section of the blood vessel. The blood vessel is, for example, the aorta, in particular in the case of a left-heart support system, or the common trunk (Truncus pulmonalis) into the two pulmonary arteries, in particular in the case of a right-heart support system, preferably the aorta. The method preferably serves to determine a fluid volume flow from a ventricle of a heart, in particular from a (left) ventricle of a heart to the aorta by a (fully) implanted, (left) ventricular (heart) support system. The fluid is regularly blood. The support system is preferably arranged at the exit of the left ventricle of the heart or the left heart chamber. The support system is particularly preferably arranged in the aortic valve position.

The support system is preferably implanted such that it is located in the fluid flow at least partially, preferably completely, or with at least 50%, particularly preferably at least 85%, or even at least 95% of its (outer) surface. Furthermore, the support system is preferably located along at least 50%, particularly preferably at least 85%, or even at least 95% of its length in the fluid flow. One end of the support system, in the region of which or on which the electric motor is located, is preferably located at least partially in the aorta. Furthermore, the opposite end of the support system, in the region of which or on which an (inlet) cannula of the support system is located, is preferably located at least partially in a ventricle (the left ventricle) of the heart. Furthermore, the support system is preferably positioned centrally in the aortic valve so that blood is drawn distally from the ventricle and dispensed proximally into the Aorta ascendens. Preferably, the support system is arranged in a blood vessel, such as an artery, in particular the aorta, at least partially, preferably completely, or with at least 20%, preferably at least 40%, particularly preferably at least 50%, or even at least 95% of its (outer) surface. The support system is particularly preferably implanted such that it is (completely) located in the (ascending or descending) aorta.

The fluid volume flow to be determined is the one that flows through the support system (itself). In other words, this relates in particular to a fluid volume flow that only flows through the support system itself. The fluid volume flow to be determined is usually the so-called pump volume flow (formula symbol Qₚ), which (only) quantifies the flow through the support system itself. The method is in particular suitable for determining the pump volume flow (Qₚ) of a (fully) implanted (left) ventricular heart support system (LVAD), in particular in the aortic valve position and/or by the support system itself.

The method is based in particular on (thermally) anemometric (measuring) principles for flow measurement. The basic principle in this case is that a flowing medium cools a hot body as a function of the flow rate. The method advantageously allows a continuous, precise measurement of Qₚ by a sensor element which is integrated into an LVAD and based on thermal anemometry. With the solution presented here, the heart-time volume (at least approximately through Qₚ) can also advantageously be provided outside the surgical scenario with comparable quality as when using a dilution catheter.

The solution proposed here is characterized in particular by an integration of one or more heating elements or one or more heating elements and one or more temperature sensors into an inlet cannula of a support system (VAD). In the method, Qₚ is advantageously calculated from the measured voltage data of at least one heating element and/or of at least one temperature sensor. In particular, three possible operating principles can be used in this case, a constant current anemometry, a constant temperature anemometry, or a pulse response method.

In step a) a fluid temperature parameter in the region of a cannula of the support system is determined. A (separate) temperature sensor can, for example, be used for the determination. The determination can alternatively or cumulatively take place by the heating element itself. For example, an electrical series resistance of the heating element can be used for this purpose. The fluid temperature parameter can be a (fluid) temperature, a temperature sensor current, a temperature sensor output (current) signal, or a (temperature-dependent) electrical resistance value, in particular of the heating element.

In step a), a temperature sensor in the region of a cannula of the support system is operated. The operation comprises in particular measuring a fluid temperature and/or changing the fluid temperature. The temperature sensor is preferably arranged on an inner surface or an outer surface of the cannula. Furthermore, at least two temperature sensors can preferably be provided. In this case, a temperature sensor can be arranged upstream of the heating element and a further temperature sensor can be arranged downstream of the heating element.

The cannula is in particular an inlet cannula, which can also be referred to as a suction tube. The (inlet) cannula is preferably configured such that, in the implanted state, it can guide fluid from a (left) ventricle of a heart to a flow machine of the support system and/or to the aorta.

The temperature sensor or the temperature sensors is/are particularly preferably arranged at a distance from the heating element. This allows the advantage that the temperature sensor is not thermally influenced by the heating element, which is in particular advantageous if the temperature sensor is a reference temperature sensor. NTC thermistors, PTC thermistors, resistance elements such as platinum, semiconductor junctions, or thermocouples can be used as temperature sensors.

The temperature sensor or a further temperature sensor can be introduced into the heating element or arranged on the heating element. If at least two temperature sensors are provided, it is preferred in this case that a reference temperature sensor is arranged at a distance from the heating element and a further temperature sensor is introduced into the heating element or arranged on the heating element. If only one temperature sensor is provided, it may be necessary that the heating element is switched off or not operated in a heating state during a measurement of a reference temperature by the temperature sensor. Positioning a flat temperature sensor between the cannula inner wall and the heating element or positioning a temperature sensor on the heating element is preferred. A particularly preferred implementation is a central positioning of the temperature sensor in the heating area of the heating element. A possible form of implementation would also be a three-layer structure, wherein a heating coil is positioned as a temperature sensor between a lower and a middle polyimide film and a platinum wire coil is positioned as a temperature sensor between the middle and an upper polyimide film.

A reference temperature of the fluid is preferably determined, in particular measured, in step a). The reference temperature is preferably determined by a reference temperature sensor, which is particularly preferably a component of the support system. The reference temperature sensor can, for example, be arranged in and/or on an (inlet) cannula of the support system. The reference temperature usually represents a background temperature of the fluid, in other words a fluid temperature, which is in particular not thermally influenced by the heating element and/or a flow machine of the support system.

In step b), a heating element which can bring about a change in a fluid temperature in the cannula is operated. In other words, this means in particular that the heating element is configured and arranged such that it can cause a change in a fluid temperature in the cannula. For this purpose, the heating element can be arranged directly inside the cannula or on an inner surface of the cannula. However, it is (alternatively) possible that the heating element is arranged in a wall of the cannula, on an outer surface of the cannula, or even at a distance from the cannula, as long as the heating element is able, for example, by means of heat conduction, to increase a fluid temperature of at least a part of the fluid inside the cannula. For operation, the heating element is generally controlled with a current.

The heating element is preferably formed with at least one heating filament or thermofilament. A heating element, in particular a round or tubular heating element, which coats the inner surface of the cannula at least in a segment region or circumferential portion and/or longitudinal portion is preferably provided. Furthermore, the heating element is preferably formed in the manner of a (flexible) heating film which at least partially coats the inner surface of the cannula. At least one heating filament is particularly preferably arranged in or on the film. Preferably, the heating filament extends (e.g., in a meandering pattern and/or in loops) in particular continuously over at least 50% of or even over the (entire) inner surface of the cannula coated by the film. At least two heating filaments may be provided. It is preferred that the heating filament or thermofilament is implemented on the wall inside the cannula (on the inside of the cannula wall), whereby a defined blood volume is advantageously investigated and heating, e.g., of the aortic valve when the support system slips can be ruled out. If more than one heating element or heating filament is provided, they can be arranged at opposite positions of the inner surface of the cannula. The heating elements or heating filaments are furthermore preferably jointly actuated or energized.

It is also advantageous if the heating element itself is used as a temperature sensor. The heating element is preferably configured both to bring about a change in a fluid temperature in the cannula and to detect, in particular measure, a change in a fluid temperature in the cannula. The heating element itself can be used as a temperature sensor, in particular by a suitable selection of the heating element, in particular the heating filament material (resistance change in case of temperature change). An advantageous embodiment of the heating element is therefore, for example, a (platinum) wire coil (heating filament made of a platinum alloy and arranged in a meandering pattern) between, for example, polyimide films or on a film. Preferably, the heating element comprises heating coils produced in the thin-film process from conductive, resistive materials (e.g., platinum alloy). In this case, the heating element can be used, for example, as a temperature sensor in that a heating element (series) resistance is measured. In order to measure the reference temperature or the fluid background temperature, the heating element (series) resistance can be measured, for example, with the heater switched off or in a phase in which the heating element is not operated in a heating state (e.g., as determined by means of a heating voltage and/or a heating current). If the heating element itself can be used as a temperature sensor, no (further or separate) temperature sensor must be provided and the heating element can in this case be operated in step a) instead of the (separate) temperature sensor. In this context, it is particularly preferred that (only) one (platinum) heating coil which can also be used as a temperature sensor is used as the heating element or in the heating element. In the switched-off state, i.e., when the heating element is not operated in a heating state, the (platinum) heating element or the heating coil could be used as a reference temperature sensor; during operation, i.e., when the heating element is operated in a heating state, it could be used as a heating element and simultaneously as an operating temperature sensor. For this purpose, a (known) temperature dependence of a heating element (series) resistance can be used, for example.

The heating element in this case is a component regularly provided in addition to an electric motor of the support system, which is in particular arranged separately from the electric motor. A heating element in this case is in particular understood to mean an electrically operable component, which preferably converts at least 70%, particularly preferably at least 80%, or even at least 90% of the electrical energy supplied to it into heat. Consequently, a heating element in this case in particular does not mean an electric motor that drives a flow machine of the support system.

In step c), the fluid volume flow is determined using at least the fluid temperature parameter or the change thereof and at least one heating element operating parameter or the change thereof. In step c), the fluid volume flow is preferably determined using at least one temperature sensor operating parameter or the change thereof and at least one heating element operating parameter or the change thereof. In other words, this means, in particular, that the fluid volume flow is determined using both a temperature sensor operating parameter or the change thereof and a heating element operating parameter or the change thereof. A heating element operating parameter can, for example, be understood to be a heating element temperature, a heating element current, or a heating element output (current) signal. A temperature sensor operating parameter can be understood to mean a temperature measured therewith, a temperature sensor current, or a temperature sensor output (current) signal. Here, a change can in particular be understood to mean a pulse, which can advantageously be transmitted by the heating element and detected by the temperature sensor.

According to a (first) advantageous embodiment, it is proposed that the heating element be operated with a defined electrical power. A temperature of the heating element can be measured in this case. This (first) embodiment relates in particular to so-called constant current anemometry. In constant current anemometry, the heating element is operated with a defined electrical power and the resulting temperature is measured.

According to a (second) advantageous embodiment, it is proposed that the heating element be kept at a constant temperature. An electrical power of the heating element can be measured in this case. This (second) embodiment relates in particular to so-called constant temperature anemometry. In constant temperature anemometry, the heating element is kept at a constant temperature and the electrical power required for this purpose is measured.

According to a (third) advantageous embodiment, it is proposed that the heating element be operated in a pulsed manner. In this case, in step c), a change in a fluid temperature can be detected by means of a temperature sensor positioned in particular downstream of the heating element. This (third) embodiment relates in particular to a so-called pulse response method.

In the pulse response method, the heating element is operated in a pulsed manner and the time is measured until the thermal pulse is measured at a downstream temperature sensor. In order to improve the measurement resolution, the pulsed operation can be carried out, for example, by means of a binary random number sequence and the time delay can be determined by an autocorrelator. Furthermore, an additional consideration of the maximum amplitude of the response pulse in the calculation is preferred.

The fluid volume flow determined in step c) is preferably provided, for example, in a step d) as a control parameter for the support system. A processing unit of the support system can provide this control parameter as an output variable, in particular to a control unit of the support system that preferably regulates the power of an electric motor and thus in particular also the (blood) delivery rate of the support system.

A further aspect proposes a processing unit configured to carry out a method proposed here. The processing unit can have a memory in which calibration data can be stored. As an alternative or in addition to the calibration data, at least one (speed-dependent) calibration factor and/or a thermal model of the heating element can also be stored in the memory. In addition, the processing unit can comprise a microprocessor which can access the memory. The processing unit preferably receives data from at least one heating element and/or at least one temperature sensor. The processing unit can furthermore comprise an electronic assembly for controlling and reading the heating element and the temperature sensor.

According to a further aspect, an implantable, vascular support system is proposed, comprising:
a temperature measuring device in the region of a cannula of the support system,
a heating element which can bring about a change in a fluid temperature in the cannula.

The support system is preferably a left-ventricular heart support system (LVAD) or a percutaneous, minimally invasive left-heart support system. Furthermore, the support system is preferably fully implantable. In other words, this means in particular that the means required for the detection, in particular the reference temperature sensor, the motor temperature sensor, and the current sensor, are completely located in the body of the patient and remain there. The support system is particularly preferably configured and/or suitable for being arranged at least partially in a ventricle, preferably in the left ventricle of a heart, and/or in an aorta, in particular in the aortic valve position.

The temperature measuring device is preferably formed with a temperature sensor. The temperature measuring device can furthermore preferably also comprise a further temperature sensor. However, it is not mandatory that the temperature measuring device is provided separately from the heating element. Rather, the temperature measuring device can also be formed in the heating element and/or by the heating element itself. An (implicit) temperature measurement based on a heating element series resistance is particularly preferred for this purpose.

Furthermore, the support system preferably comprises a flow machine, such as a pump. The support system preferably has an electric motor. The electric motor is regularly a component of the flow machine. The support system is preferably elongated and/or tubular. Preferably, a (n) (inlet) cannula and a flow machine are arranged in the region of opposite ends of the support system.

According to an advantageous embodiment, the support system furthermore comprises a processing unit configured for carrying out a method proposed here.

The details, features, and advantageous embodiments discussed in connection with the method can also arise accordingly in the processing unit and/or the support system presented here and vice versa. In this respect, reference is made in full to the explanations there regarding the detailed characterization of the features.

The solution presented here as well as its technical environment are explained in more detail below with reference to the figures. It should be pointed out that the invention should not be limited by the exemplary embodiments shown. In particular, unless explicitly stated otherwise, it is also possible to extract partial aspects of the facts explained in the figures and to combine them with other components and/or insights from other figures and/or the present description. The following are shown schematically:

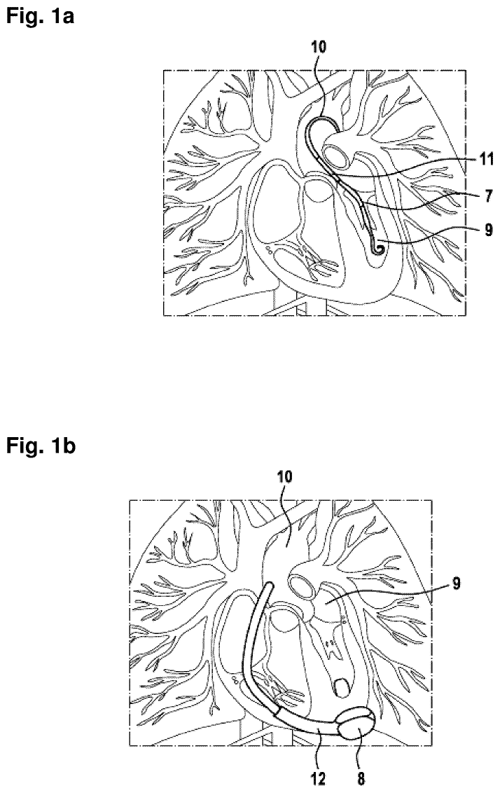

FIG. 1a a percutaneous, minimally invasive left-heart support system,

FIG. 1b a left-heart support system invasively implanted under the chest opening,

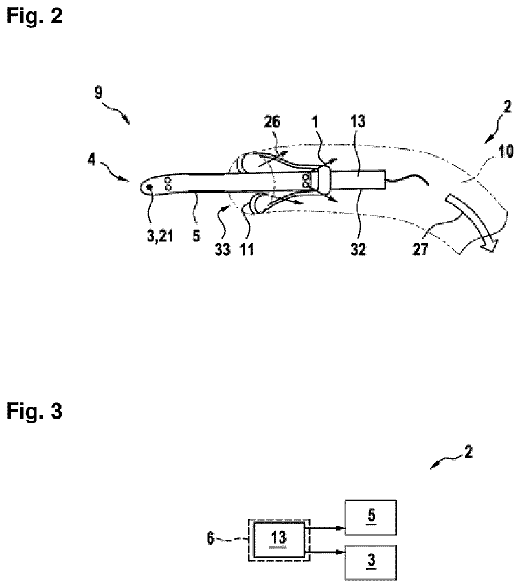

FIG. 2 an implanted vascular support system that can carry out a constant current and constant temperature method,

FIG. 3 a component architecture of a support system according to FIG. 2,

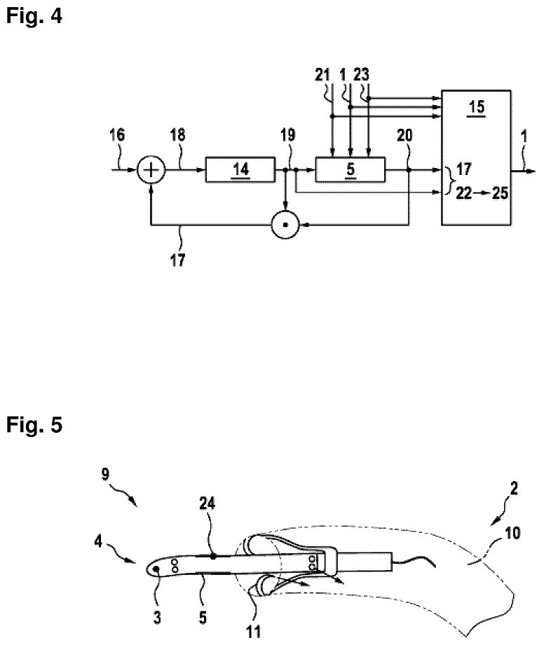

FIG. 4 an illustration of a control circuit of a support system according to FIG. 2,

FIG. 5 a further implanted vascular support system that can carry out a constant current and constant temperature method,

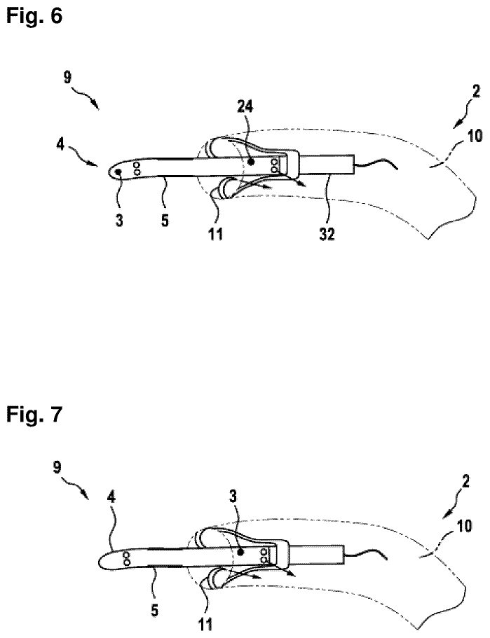

FIG. 6 a further implanted vascular support system that can carry out a pulse response method,

FIG. 7 a further implanted vascular support system that can carry out a pulse response method, and

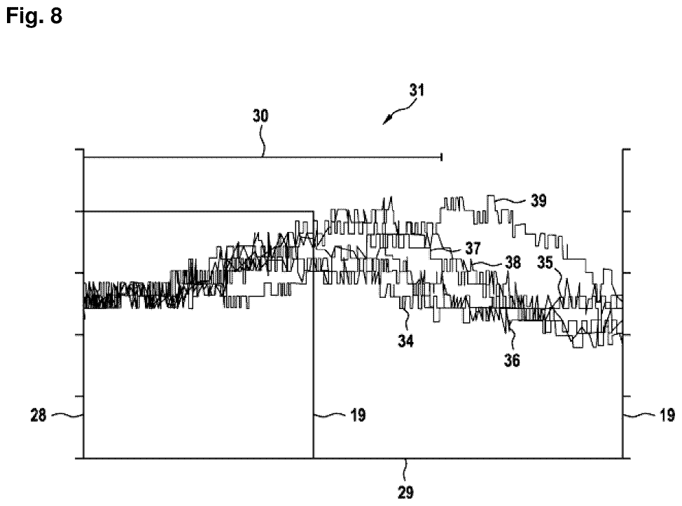

FIG. 8 measured value time curves for the support system according to FIG. 6 or FIG. 7.

Implanted left-heart support systems (LVAD) exist mainly in two design variants, as shown in FIGS. 1a and 1b. FIG. 1a shows a (percutaneous) minimally invasive left-heart support system 7, while FIG. 1b shows an apical left-heart support system 8 invasively implanted under the chest opening. The variant according to FIG. 1a conveys blood directly from the left ventricle 9 into the aorta 10, since the (percutaneous) minimally invasive left-heart support system 7 is positioned centrally in the aortic valve 11. The variant according to FIG. 1b conveys the blood apically from the left ventricle 9 via a bypass tube 12 into the aorta 10.

FIG. 2 schematically shows, in the aortic valve position, an implanted vascular support system 2 that can carry out a constant current and constant temperature method.

The support system 2 is here, by way of example, a left ventricular heart support system (LVAD), a tubular elongated structure with a cannula portion, in which a (n) (inlet) cannula 4 is formed, and with a flow machine portion which is connected to the cannula portion and in which a flow machine 32 is arranged. The support system 2 protrudes from the aorta 10 through the aortic valves 11 distally into the ventricle 9. The (inlet) cannula 4 of the support system 2 protrudes into the ventricle 9. A fluid volume flow 1 is conveyed, for example pumped, from the ventricle 9 through the cannula 4 into the aorta 10 using the flow machine 32 (e.g., a pump that can have an electric motor) of the support system 2. Therefore, the fluid volume flow 1 is also referred to as the pump volume flow (Qₚ), which only quantifies the flow through the support system 2 itself.

In addition, it can be seen in FIG. 2 that a certain aortic valve volume flow 26 reaches the aorta 10 via the physiological path through the aortic valves 11. The heart-time volume or the total fluid volume flow 27 (Q_{HTV}) passing through a cross-sectional geometry 33 of the aorta 10 in the region of the support system 2 from the ventricle 9 to the aorta 10 is therefore the sum of fluid volume flow 1 (Qₚ) and aortic valve volume flow 26 (Qₐ).

A temperature sensor 3 is arranged in the region of the cannula 4. For this purpose, the temperature sensor 3 is positioned, by way of example, on the distal end of the cannula 4 (in the ventricle 9, from where the fluid, e.g., blood, flows). The support system 2 furthermore comprises a heating element 5 which can bring about a change in a fluid temperature in the cannula 4, e.g., by Joule heating or ohmic resistance heating, when the heating element 5 is energized.

The temperature sensor 3 according to FIG. 2 is a reference temperature sensor, which detects a reference temperature 21, which, by way of example, is in this case the background blood temperature. For this purpose, the (reference) temperature sensor 3 is positioned in the thermally uninfluenced blood flow upstream of the heating element 5, which represents a heat source, here by way of example in the region before or upstream of the heating element 5. Instead of a separate (reference) temperature sensor 3, the value of a further (second) temperature sensor arranged, for example, at the height of the heating element 5 or downstream thereof (cf. FIGS. 5, 6: reference sign 24; FIG. 7: reference sign 3) can also be used if the system is not in operation and this further temperature sensor is thus not influenced by the heating element 5. Since the blood temperature changes only slowly in resting patients, this value can also represent a good estimation of the background temperature. In addition, depending on the design of the heating element 5, the electrical resistance of the heating element 5 itself can also be used as a temperature sensor 3.

If a separate reference temperature sensor is used, as is the case with the temperature sensor 3 according to the illustration in FIG. 2, this separate reference temperature sensor should be positioned in the support system 2 in such a way that it is not influenced by the thermal output of the heating element 5, e.g., at the tip of the support system 2 pointing toward the ventricle 9 and/or the cannula 4 and/or in a thermally decoupled manner upstream (with respect to the blood flow) of the heating element 5. As a result, it is advantageously possible to determine the temperature rise by thermal energy supply into the observed fluid volume. Due to the directed flow in the medium, an exemplary minimum distance of the reference temperature sensor to the heating element 5 is determined in particular (mainly) from the thermal conductivity of the carrier material. Distances of at least 5 mm [millimeters] are advantageous for non-metallic carrier material.

The operating principle here is based on sufficiently knowing the thermal capacity (formula symbol C; cf. reference sign 23 in FIG. 4) of the fluid, in this case blood, and on determining the electrical power dQ necessary to heat the blood by a defined temperature dT:

$$C = \frac{dQ}{dT}$$

With a sufficiently known thermal capacity C (provided in the algorithm), measured energy supply dQ, and temperature rise dT determined from two measured (fluid) temperatures, the fluid volume V or the fluid volume flow 1 (formula symbol Q) transferred in the observational period can thus be calculated. The background blood temperature required for the difference dT can in this case be calculated either by means of a (reference) temperature sensor 3 or from the value of a further temperature sensor (cf. explanations above) if the heating element was not active for a sufficiently long time.

The heating element 5 is in this case formed, by way of example, with a heating filament or thermofilament. The thermofilament is implemented on the wall inside the cannula 4, which can also be referred to as a suction tube, as a result of which a defined blood volume is advantageously investigated and heating, e.g., of the aortic valve 11 when the support system slips can be ruled out.

Regarding the operating mode of the embodiment according to FIG. 2, reference is also made to the following explanations regarding FIG. 4.

FIG. 3 schematically shows a component architecture of a support system according to FIG. 2. In this case, the support system 2 comprises, by way of example, a control unit 13, a temperature sensor 3, and a heating element 5 formed, by way of example, as a thermofilament or heating filament. By way of example, the control unit 13 is here a component of a processing unit 6 of the support system 2.

FIG. 4 schematically shows an illustration of a control circuit of a support system 2 according to FIG. 2. The reference signs are used uniformly so that reference is also made to FIGS. 2 and 3 with respect to the explanation of the operating mode of the embodiment according to FIGS. 2 to 4.

The exemplary control circuit shown in FIG. 4 can be implemented in the control unit 13 according to FIG. 3, which in turn can be a component of the support system 2, in particular of a processing unit 6 of the support system 2. The control circuit comprises a controller 14 and the heating element 5. The disturbance variables influencing the heating element 5 (control path) are the reference temperature 21, the fluid volume flow 1, and the thermal capacity 23 (of the fluid, here blood). The control variable here is the current 20 and is returned to the controller 14. Here, the current 20 (control variable) and the voltage 19 (manipulated variable) are returned together by means of the determined actual power 17. The control deviation 18 results from a subtraction of the actual power 17 from the target power 16. The aforementioned disturbance variables of fluid volume flow 1, reference temperature 21, and thermal capacity 23 as well as the current 20 (control variable) and the voltage 19 (manipulated variable) are also provided to a computing unit 15, which determines the actual power 17 from the voltage 19 and the current 20 as well as the actual electrical resistance 22 of the heating element 5 and also determines the heating element temperature 25 from the actual electrical resistance 22 (e.g., based on the known temperature dependence of the resistance). The computing unit 15 calculates therefrom the fluid volume flow 1, wherein the latter can be provided as an average volume flow.

In the embodiment as constant current anemometry, the heating element 5 is here, by way of example, supplied with constant power by the controller 14 in the control unit 13 and both the electrical resistance 22 for measuring the heating element temperature 25 and the reference temperature 21 is read from the reference temperature sensor 3 (or heating element resistance 22 when the heater is switched off (i.e., the heating element 5 is not operated in a heating state) in order to determine the reference temperature 21). The fluid volume flow 1 or of Qₚ is calculated in the computing unit 15 on the basis of the electrical heating element power consumption 17, the heating element temperature 25 determined on the basis of the electrical resistance 22 of the heating element 5, and the reference temperature 21.

In the embodiment as constant temperature anemometry, the heating element temperature 25 of the heating element 5 is here, by way of example, kept at a defined temperature by the controller 14 or at a defined temperature rise based on the reference or background temperature 21. On the basis of the required filament power consumption 17 and the background temperature 21, the fluid volume flow 1 or Qₚ is calculated in the computing unit 15 of the control unit 13.

FIG. 5 schematically shows a further implanted, vascular support system 2, which can carry out a constant current and constant temperature method. The support system 2 according to FIG. 5 has many common features with the support system 2 according to FIG. 2, so that reference is made in this respect to the above explanations regarding FIG. 2. The design variant according to FIG. 5 differs from that according to FIG. 2 in that a further (second) temperature sensor 24 is positioned so as to be thermally coupled to the heating element 5 so that the temperature of the heating element 5 can be determined not based on the electrical resistance 22 of the heating element 5, but based on the electrical resistance of the additional temperature sensor 24.

FIG. 6 schematically shows a further implanted, vascular support system 2, which can carry out a pulse response method. In this variant, a further temperature sensor 24 arranged preferably on the wall inside the cannula 4 is spaced apart from the heating element 5 (in the direction of the flow machine 32, downstream of the heating element 5) so that transit time and thermal dilution effects can be observed. As with the design variants described above, an optional (cf. FIG. 7) reference temperature sensor, which is formed here by the temperature sensor 3, is positioned upstream in order to determine the reference or background temperature 21 of the fluid (here: blood). In this case, it is ensured that the temperature sensor 3 and the additional temperature sensor 24 are thermally decoupled from the heating element 5 and that the further temperature sensor 24, due to its spatial proximity to the flow machine 32, is also thermally decoupled therefrom. Depending on the carrier material, a distance of 5-10 mm is good in this case.

The heating element 5 is subjected to a power pulse 31 and introduces a defined amount of energy Eₚ into the blood volume of the cannula 4, which leads to an increase in the blood temperature. Due to the (pump) activity of the flow machine 32, the blood flows with a Qₚ-dependent flow rate further in the direction of the further temperature sensor 24, which observes a temperature maximum Tₘ after a Qₚ-dependent transit time Δt. On the basis of Eₚ or the heating element power consumption 17, with Δt, the reference temperature 21, and Tₘ, the fluid volume flow 1 or Qₚ is calculated in the control unit 13 (transit time Δt or transit time Δt and amplitude height Tₘ).

The observable effects are both a transit time, wherein a high fluid volume flow 1 corresponds to a short transit time from the heating element 5 to the further temperature sensor 24, and, based on the fixed thermal resistance of the heating element 5 to the blood volume and the fixed thermal capacity 23 of the blood, an amplitude change, wherein a slow fluid volume flow 1 corresponds to a sharp temperature increase at the further temperature sensor 24 and a fast flow corresponds to a small temperature increase.

FIG. 7 schematically shows a further implanted, vascular support system 2, which can carry out a pulse response method. The support system 2 according to FIG. 7 has many common features with the support system 2 according to FIG. 6 so that reference is made in this respect to the above explanations regarding FIG. 6. The difference is that only one temperature sensor 3 is provided in FIG. 7. This temperature sensor is preferably on the wall inside the cannula 4 and in this case fulfills the purpose which the further temperature sensor 24 fulfills in the embodiment according to FIG. 6. The embodiment according to FIG. 7 thus manages without a (separate) reference temperature sensor.

FIG. 8 shows schematically measured value time curves for the support system 2 according to FIG. 6 or FIG. 7. Temperature curves measured by means of the temperature sensor arranged downstream of the heating element 5 (reference sign 24 in FIG. 6 and reference sign 3 in FIG. 7) are plotted over the time 29, wherein the temperature was measured as a voltage value via an analog/digital converter so that both the voltage 19 and an analog/digital converter output 28 are plotted over the time 29. Various measured value curves are plotted, namely a first measured value curve 34, a second measured value curve 35, a third measured value curve 36, a fourth measured value curve 37, a fifth measured value curve 38, and a sixth measured value curve 39, wherein the measured value curves are arranged according to decreasing fluid volume flow (pump volume flow); measured value curve 39 thus represents the temperature curve at the temperature sensor in the case of a low fluid volume flow and measured value curve 34 thus represents the temperature curve at the temperature sensor in the case of a high fluid volume flow. In addition, the time difference 30 until the pulse 31 of the measured value curve 39 was measured is marked only as an example. It can be clearly seen that the time difference 30 is inversely proportional to the fluid volume flow, as is the amplitude (the maximum) of the measured value curve. In addition, in the illustration according to FIG. 8, the pulses 31 of the further measured value curves 34, 35, 36, 37, and 38 can also be seen, thus a total of six pulses 31. For the explanation of the measured value curves, reference is made to the above explanations regarding FIGS. 6 and 7, in particular to the observed effects described there.

The solution proposed here allows in particular one or more of the following advantages:
By integrating the sensor into the inlet cannula of the VAD, contact between the tissue and the heating element is prevented, thereby preventing tissue damage.
The integration into the inlet cannula has the advantage that the (flow) geometry and thus the investigated blood volume is known, which simplifies or replaces the calibration of the sensor depending on the implementation variant. Commercially available catheters require the administration of an ice-water bolus in order to calibrate with respect to the blood vessel volume.
Continuous Qₚ measurement allows rapid diagnosis of suction, i.e., suction of the inlet tube to the ventricle wall, whereby the pump function is impaired.

In summary, the following preferred features of the invention should in particular be noted:

A method for determining a fluid volume flow 1 through an implanted vascular support system 2 comprises the following steps:
a) determining a fluid temperature parameter in the region of a cannula (4) of the support system (2),
b) operating a heating element (5) which can bring about a change in a fluid temperature in the cannula (4),
c) determining the fluid volume flow (1) using at least the fluid temperature parameter or the change thereof and at least one heating element operating parameter or the change thereof.

An implantable vascular support system, i.e., a vascular support system that can be arranged in the human or animal body, contains a temperature measuring device in the region of a cannula 4 of the support system 2 and comprises a heating element 5 which can bring about a change in a fluid temperature in the cannula (4).

1-22. (canceled)

23. A cardiac support system comprising:
a flow machine configured to convey blood through a cannula from a distal inlet of the cannula toward the flow machine;
a temperature sensor arranged to measure a reference temperature of blood at the distal inlet of the cannula; and
a heating element located on or within an inner wall of the cannula and positioned downstream of the temperature sensor with respect to a direction of blood flow from the distal inlet toward the flow machine, the heating element being configured to change a temperature of blood flowing in the cannula.

24. The cardiac support system of claim 23, wherein the heating element comprises a flexible heating film that at least partially coats an inner surface of the cannula along a circumferential portion and/or a longitudinal portion.

25. The cardiac support system of claim 23, wherein the heating element includes a thin-film heating coil formed of a conductive, resistive material.

26. The cardiac support system of claim 25, wherein the thin-film heating coil is produced in a thin-film process from a platinum-based resistive material.

27. The cardiac support system of claim 23, wherein the heating element comprises at least one heating filament extending in a meandering pattern and/or in loops along an inner surface of the cannula.

28. The cardiac support system of claim 24, wherein the flexible heating film coats at least 50% of the inner surface of the cannula.

29. The cardiac support system of claim 23, wherein the temperature sensor is positioned at least 5 mm upstream of the heating element.

30. The cardiac support system of claim 23, further comprising a second temperature sensor positioned downstream of the heating element and configured to detect a temperature change in blood flowing past the heating element.

31. The cardiac support system of claim 23, further comprising a temperature sensor thermally coupled to the heating element and configured to detect a temperature of the heating element.

32. The cardiac support system of claim 23, further comprising one or more hardware processors configured to apply a constant power to the heating element.

33. The cardiac support system of claim 23, further comprising a controller configured to keep the heating element at a defined temperature or at a defined temperature range above the reference temperature measured at the distal inlet.

34. The cardiac support system of claim 23, further comprising a processing unit configured to determine a temperature of the heating element based on an electrical resistance of the heating element.

35. The cardiac support system of claim 30, wherein the heating element is operated in a pulsed manner and the cardiac support system is configured to detect a transit time for a temperature maximum at the second temperature sensor.

36. A cardiac support system comprising:
a flow machine configured to convey blood through a cannula from a distal inlet of the cannula toward the flow machine;
a temperature sensor arranged to measure a reference temperature of blood at the distal inlet of the cannula;
a heating element located on or within a wall of a cannula and positioned downstream of the temperature sensor with respect to a direction of blood flow from the distal inlet toward the flow machine, the heating element being configured to change a temperature of blood flowing in the cannula; and
a processing unit configured to operate the flow machine based on the reference temperature and an operating parameter of the heating element.

37. The cardiac support system of claim 36, wherein the heating element is embedded within the wall of the cannula and comprises a multilayer structure including a polymeric film stack and a resistive wire coil.

38. The cardiac support system of claim 36, wherein the processing unit is configured to adjust operation of the flow machine to prevent suction events based on the reference temperature and the operating parameter of the heating element.

39. The cardiac support system of claim 36, wherein the heating element extends along a circumferential portion and/or a longitudinal portion of a wall of the cannula.

40. The cardiac support system of claim 36, further comprising a second temperature sensor positioned downstream of the heating element and configured to detect a temperature change in blood flowing past the heating element.

41. The cardiac support system of claim 40, wherein the second temperature sensor is positioned 5 to 10 mm downstream of the heating element.

42. The cardiac support system of claim 36, wherein the cannula is configured to guide blood from a ventricle of a heart into an aorta, and the distal inlet is positioned in the ventricle.

43. The cardiac support system of claim 36, wherein the heating element comprises a flexible heating film that is round or tubular and conforms to an inner surface of the cannula.

44. The cardiac support system of claim 36, further comprising a processing unit configured to determine a temperature of the heating element based on an electrical resistance of the heating element.
